(19) United States
(12) Patent Application Publication
(10) Pub. No.: US 2026/0146019 A1
(43) Pub. Date: May 28, 2026

(54) ESTERIFICATION PROCESSES USING HOMOGENEOUS ACID CATALYSTS

(71) Applicant: New Product Innovation, LLC, Johnston, IA (US)

(72) Inventors: Louis Kapicak, Cross Lanes, WV (US); David James Schreck, Lake City, MN (US)

(73) Assignee: New Product Innovation, LLC, Johnston, IA (US)

(21) Appl. No.: 19/399,167

(22) Filed: Nov. 24, 2025

Related U.S. Application Data
(60) Provisional application No. 63/723,808, filed on Nov. 22, 2024.

(51) Int. Cl. C07C 67/08; C07C 67/54

(52) U.S. Cl. CPC C07C 67/08; C07C 67/54

(57) ABSTRACT

Disclosed herein are various embodiments that pertain to esterification processes are provided that enable the homogeneous, acid catalyst to be rendered into a less, acidic, liquid phase from which the esterification product can be separated by vaporization. It is believed that in the processes of this disclosure, the homogeneous, acidic catalysts used for the esterification are converted to less acidic, stable, acylated species that remain in the liquid phase from which the esterification product can be recovered by vaporization.

### CROSS-REFERENCES & RELATED APPLICATIONS

This application claims priority to U.S. Provisional Application No. 63/723,808 filed Nov. 22, 2024, and entitled “ESTERIFICATION PROCESSES USING HOMOGENEOUS ACID CATALYSTS,” which is hereby incorporated by reference in its entirety under 35 U.S.C. § 119(e).

### TECHNICAL FIELD

The disclosure pertains to esterification processes using homogeneous acid catalysts, and particularly to such processes wherein separation of the catalyst from the esterification product is facilitated. This disclosure also pertains to processes for making a homogeneous, acid catalyst having desirable catalytic activity for esterification processes that can be readily recovered from the esterification product and recycled to the esterification process.

### BACKGROUND

Esterification of carboxylic acids with alcohols using homogeneous, acid catalysts is well known. Homogeneous, acid catalysts provide advantages including the ability to disperse the catalyst in the reaction medium. The challenge, however, is that the esterification product must be separated from the acid catalyst. Frequently, the separation of the homogeneous, acid catalyst is by neutralization with a base and precipitation of the corresponding salt. The procedure requires a solids separation operation such as filtration or centrifugation, which add to the cost of making the esterification product. Care also needs to be taken to avoid a difficult to separate neutralization product such as a gelatinous structure. Moreover, the generation of solids increases the risk of solids build-up and fouling of equipment. Accordingly, an interest exists in providing a method for separating the homogeneous, acid catalyst from the esterification product that does not require the generation of solids and the attendant solids separation unit operations.

### BRIEF SUMMARY

According to various embodiments in this disclosure, esterification processes are provided that enable the homogeneous acid catalyst to be rendered into a less acidic liquid phase from which the esterification product can be separated by vaporization. Without wishing to be limited to theory, it is believed that in the processes of this disclosure, the homogeneous, acidic catalysts used for the esterification are converted to less acidic, stable, acylated species that remain in the liquid phase from which the esterification product can be recovered by vaporization. Additionally, in many instances, the processes of this disclosure provide a reduction of high molecular weight compounds, also known as heavies. Again, without wishing to be limited by theory, it is believed that in the processes of this disclosure, precursors to the formation of heavies are converted to more stable species.

The broad aspects of this disclosure pertain to processes for the esterification of carboxylic acid having at least one carboxylic acid moiety and alcohol having at least one hydroxyl moiety to provide an ester, which processes comprise:
a. contacting in a liquid phase the carboxylic acid and alcohol under esterification conditions including the presence of a catalytically-effective amount of homogeneous, acidic catalyst having an oxy-sulfur-containing or oxy-phosphorus-containing anion whereby water and ester are produced;
b. removing water produced or introduced during step (a) to provide a reaction product comprising ester, and homogeneous, acidic catalyst that contains less than about 5 mass percent water; in some embodiments, the acidic catalyst may contain less than about 2 mass percent water;
c. contacting under acylation conditions the reaction product having water removed with carboxylic anhydride in an amount sufficient to convert at least about 90 (at least about 98 in some embodiments) mass percent, an in certain embodiments substantially all, of the acidic catalyst to acylated catalyst and provide a treated reaction product comprising ester carboxylic acid co-product; and
d. separating ester from the treated reaction product by vaporization to provide a residual liquid phase comprising acylated catalyst.

Since esterification is an equilibrium reaction, the reaction product, in addition to some water, will contain carboxylic acid and alcohol. Typically, the carboxylic anhydride also reacts with water and alcohol, especially in the presence of the acidic catalyst, to provide carboxylic acid and carboxylic ester, wherein the carboxyl moieties are from those of the carboxylic anhydride. Consequently, the amount of carboxylic anhydride provided in step (c) should reflect those reactions. Without wishing to be limited by theory, it is believed that the presence of alcohols in the product, especially at the higher temperatures required to recover the ester by vaporization, react to form heavies. The processes of this disclosure significantly reduce the presence of alcohols through reaction with the carboxylic anhydride, often to fewer than 0.2 parts by mass of alcohol per 100 parts by mass of ester, and hence, the production of heavies is reduced.

In some embodiments, where the residual liquid phase of step (d) contains heavies, step (d) is conducted in at least two steps. The first step can be a vaporization the ester to provide a concentrated residual liquid phase in which the mass of the treated product is reduced to less than 25 percent of its original mass. The second step can be vacuum vaporization of the ester at a temperature less than about 270° C. and an absolute pressure less than about 10 kPa to provide a further concentrated residual liquid phase in which the mass of the treated product is reduced to less than 10 percent of its original mass. The concentrated residual liquid phase and the further concentrated residual liquid phase comprise heavies, ester and acylated catalyst. In some embodiments, at least a portion of the acylated catalyst is separated from the residual liquid phase that has had its mass reduced by vaporization of ester to less than 10 percent, optionally less than about 5 percent of the mass of the treated product. One convenient method for separation of the acylated catalyst is by extraction with water to provide an aqueous phase containing acylated catalyst or hydrolysis product thereof and an organic phase containing heavies. The separated acylated catalyst or hydrolysis product, if desired, can be recycled to step (a) as a portion of the homogeneous, acidic esterification catalyst. The carboxylic acid co-product of step (c) can be recovered

The acylated acidic catalyst produced by reaction with the carboxylic anhydride is in the liquid phase and is stable under vaporization conditions for recovering the ester. In some embodiments, essentially all of the homogeneous, acidic catalyst is reacted with carboxylic anhydride. Generally, oxy-sulfur and oxy-phosphorus acidic catalysts are subject to degradation under many of the desired esterification conditions, especially at temperatures above 100° C., and under conditions for the vaporization of the ester for product recovery. For example, sulfuric acid can provide sulfur trioxide, which would be vaporized with the ester. The presence of such degradation products in the ester is often undesirable as they must be removed from the ester to meet quality specifications. The stability of the acylated acidic catalyst substantially prevents such degradation products from forming. In certain aspects of this disclosure, e.g., using oxy-sulfur-containing anion acidic catalysts, the vaporized ester contains less than 10 (in certain embodiments less than about 1) parts per million by volume of sulfur-containing degradation products based on the volume of ester vaporized.

The carboxylic moieties of the carboxylic anhydride can be the same or different from those of the carboxylic acid reactant. In one mode, the carboxylic moieties of the carboxylic anhydride are of a different molecular weight than those of the carboxylic acid reactant, e.g., higher or lower molecular weight, so that the carboxylic acid formed from the moieties on the anhydride can be more easily separated from the ester. Where the carboxylic moieties of both the carboxylic acid reactant and the carboxylic anhydride are the same, the coproduct carboxylic acid can be handled in the same manner as the unreacted carboxylic acid reactant in the esterification reaction product.

In some embodiments, the carboxylic acid reactants have up to about 8 carbon atoms, and in other embodiments between about 2 and about 6 carbon atoms. In various embodiments, the carboxylic reactants have a normal boiling point of below about 200° C., below about 180° C. The carboxylic acid reactant can be aliphatic or can contain an aryl moiety such as benzoic acid. In some embodiments, the carboxylic acid reactants have no more than two carboxyl moieties, and most often, only one carboxyl moiety. in various embodiments, the alcohol reactants have up to 10 carbons, and in other embodiments between 1 and about 7, carbons. Optionally, the alcohols have a normal boiling point of below about 200° C., in some embodiments below about 180° C., and have one or two hydroxyl moieties. The alcohols can be aliphatic or contain an aryl moiety.

The carboxylic anhydride, as mentioned above, can have a different carboxylic moiety than the carboxylic acid reactant. In some embodiments, the carboxylic anhydride has carboxylic moieties each with up to 20, in some embodiments between 2 and 18, carbon atoms. The carboxylic anhydride can be aliphatic or can contain one or more aryl moieties.

It has been found by this disclosure that sulfuric acid acts differently when contacted with carboxylic anhydride under the acylation conditions of step (c), and ultimately results in a compound other than carboxysulfates being formed. This compound is believed to be sulfocarboxylic acid, and the compound is stable and is effective as an esterification catalyst. In another broad aspect of this disclosure, processes are provided for making sulfocarboxylic acid comprising contacting sulfuric acid and carboxylic anhydride under acylation conditions, in some embodiments at a temperature between about 80° C. and 270° C., and in other embodiments between about 100° C. or 120° C. and 250° C., in the liquid phase to produce sulfocarboxylic acid and carboxylic acid. Under the acylation conditions, any water present will react with the carboxylic anhydride to make carboxylic acid. Hence, the reactions to produce the sulfocarboxylic acid are conducted in a relatively anhydrous medium. Other liquids can be present and can serve as solvents. For instance, in the context of making esters, the liquids can be heavies, the ester and carboxylic acid. In various embodiments where a solvent is to be provided, it may be substantially non-reactive. Carboxylic acids are particularly useful as solvents. While multiple embodiments are disclosed, still other embodiments of the disclosure will become apparent to those skilled in the art from the following detailed description, which shows and describes illustrative embodiments of the disclosure. As will be realized, the disclosure is capable of modifications in various obvious aspects, all without departing from the spirit and scope of the disclosure. Accordingly, the drawings and detailed description are to be regarded as illustrative in nature and not restrictive.

### DETAILED DESCRIPTION

All patents, published patent applications, and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3, 0.63, 0.815, and so forth) are included as are all intermediate ranges (e.g., 0.2-0.5, 0.54-0.913, and so forth).

The use of the terms “a” and “an” is intended to include one or more of the elements described.

An acylated acidic catalyst is an oxy-phosphorus-containing acid or an oxy-sulfur-containing acid that is reacted with carboxylic anhydride. While the reaction product is believed to contain an acyl moiety from the reaction, the term acylated acidic catalyst is intended to cover all reaction products of the acidic catalyst with carboxylic anhydride regardless of whether or not they contain an acyl moiety and thus an acylated acid catalyst includes sulfocarboxylic acid.

An alcohol is an organic compound containing at least one hydroxyl on an aliphatic carbon.

Heavies are organic compounds having a higher normal boiling point than the ester product. The term heavies as used herein is not intended to include the acylated acidic catalyst. Heavies include compounds coproduced under one or more of esterification conditions, acylation conditions and the conditions for vaporization of the ester.

An oxy-phosphorus-containing acid comprises an anion having at least one double bond oxygen to a phosphorus atom and at least one oxygen atom of an acid group bonded to the phosphorus atom. Examples of oxy-phosphorus-containing acids include, but are not limited to, phosphoric acid and phosphonic acid.

An oxy-sulfur-containing acid comprises an anion having at least one double bond oxygen to a sulfur atom and at least one oxygen atom of an acid group bonded to the sulfur atom. Examples of oxy-sulfur-containing acids include, but are not limited to, sulfuric acid, sulfonic acid and sulfocarboxylic acid such as sulfoacetic acid.

Reactive distillation is a process in which reaction and separation occur simultaneously on some or all of the stages of a distillation column, and reaction products (e.g., ester and water) are separated and removed. The continuous removal of the reaction products via the distillation can increase conversion beyond that of the equilibrium composition in a batch reactor.

### Reactants

Examples of carboxylic acids include monoacids such as formic acid, acetic acid, propionic acid, isopropanoic acid, butyric acid, isobutyric acid, pentanoic acid, isopentanoic acid, cyclohexanoic acid, hexanoic acid, and benzoic acid; dicarboxylic acids such as oxalic acid, malonic acid, succinic acid, and terephthalic acid. Alcohols include monoalcohols such as methanol, ethanol, propanol, butanol, isobutanol, pentanol, isopentanol, hexanol, cyclohexanol, benzyl alcohol; and diols such as ethylene glycol, propylene glycol, 1,3-propanediol, 1,4-butanediol, 1,2-butanediol, and 1,3-pentanediol.

### Esterification

In various processes of this disclosure, carboxylic acid and alcohol are esterified using certain homogeneous, acidic catalysts, namely oxy-sulfur-containing acid and oxy-phosphorus-containing acid. Esterification conditions are quite well known, and the esterification conditions are not critical to the broad aspects of this disclosure. The esterification is conducted in the liquid phase, and often the liquid phase is provided by the reactants and ester products, although substantially inert solvents and dispersants could find application.

Esterification is an equilibrium reaction, and the relative concentrations of the reactants and the presence of water from the condensation reaction all have effects on the driving force to the ester. Hence the molar ratio of the reactants can be varied widely, e.g., from 100 or more:1 to 1:100 or more of carboxylic acid:alcohol. Water, for instance, can be removed from the menstruum as the esterification proceeds to assist in enhancing the driving force to the ester. From a practical standpoint, for commercial operations, the ratio of the reactants is maintained close to that required to provide a hydroxyl group for each carboxylic acid group in the feed. It is to be understood that one of the reactants may be more susceptible to loss during the esterification, such as by vaporization, and that reactant may be supplied in excess. Often the ratio of hydroxyl groups to carboxylic acid groups is in the molar range of about 0.5:1 to 2:1, optionally about 0.75:1 to 1.3:1.

Esterification conditions typically include a pressure in the range of about 10 to 500, often 50 to 250, kilopascals absolute and temperatures in the range of about 80° C. to 250° C., in some embodiments about 90° C. to about 200° C. As the removal of water enhances the driving force to produce the ester, an entrainer or azeotroping agent can be used to facilitate water removal. The duration of the esterification reaction is for a time sufficient to achieve the desired conversion to ester, taking into account that it is an equilibrium reaction.

A catalytically effective amount of catalyst is used. The concentration of the homogeneous, acidic catalyst can vary widely, and in general, the greater the concentration of catalyst, the faster the esterification reaction approaches equilibrium. The catalysts have a pKa of less than about 3, and in general, the lower the pKa, the lower the catalyst concentration needs to be to achieve a given reaction rate. As the catalysts used in this disclosure are homogeneous, desirable rates of reaction can be achieved with low concentrations of catalyst. Often, the catalyst concentration is in the range of about 0.01 to 10 grams of catalyst per 100 grams of alcohol.

The process can be batch, semi-continuous or continuous. Any suitable equipment for esterification can be used such as pipe reactors, stirred reactors including continuous stirred tank reactors, jet loop reactors and the like. For commercial operations, continuous processes using reactive distillation may be used, as water can be removed during the reaction.

In various embodiments, water is removed from the esterification reaction mixture. The water removal may be accomplished at least partially during the esterification reaction. As the carboxylic anhydride reacts with water, it is desired to provide a reaction product containing less than about 5, optionally less than about 2, mass percent water. In some instances, water is present in less than 1, in some embodiments in the range of about 0.01 to about 0.5, mass percent of the reaction product.

### Acylation

The reaction product having water removed is contacted under acylation conditions with carboxylic anhydride in an amount sufficient to convert at least about 98 percent, and in some embodiments substantially all, the acidic catalyst to acylated acidic catalyst and provide carboxylic acid co-product. As the reaction product contains unreacted hydroxyl groups, e.g., on alcohol or partially reacted diol in the case that a diol is used as the alcohol, and potentially water, some of the carboxylic anhydride will react with these hydroxyls and water. Accordingly, the quantity of carboxylic anhydride to be contacted with the reaction product should take into account that required for these reaction as well as that required for the acylation of the acidic catalyst. With respect to the acidic catalyst, the amount of carboxylic anhydride provided should be sufficient to react with all acid moieties on the acidic catalyst. The carboxylic anhydride is provided in an amount of at least 90, in some embodiments at least 98, and in other embodiments between 98 and 150, percent of that required on a stoichiometric basis to react with the hydroxyl groups, water and acid groups in the reaction product.

Copending United States patent application with attorney docket number 9022034-194046, filed on even date herewith, discloses an esterification of monoethylene glycol with acetic acid which is operated such that 2-hydroxyethyl acetate still remains in the esterification product and acetic anhydride added to react the 2-hydroxyethyl acetate to ethylene glycol diacetate. The application provides that acetic anhydride can also react with water and catalyst present.

The reaction of the carboxylic anhydride with the acidic catalyst proceeds quickly, especially at the temperatures used for esterification. Often, the contact time is less than about 1 hour, in some embodiments from about 0.01 to about 0.5 hour. The temperature of the contacting is often in the range of about 80° C. to about 250° C. and is in the liquid phase. The contact conditions are such that substantially all of the acylated acidic catalyst remains in the liquid phase.

### Recovery of the Ester

The product ester is removed from the acylated acidic catalyst and any heavies present by vaporization. Since the acylated acidic catalyst is stable and remains in the liquid phase, temperatures as high as about 250° C. or more are sometimes acceptable for vaporization of the ester. In many instances, the removal of the ester provides an acylated acidic catalyst bottoms fraction in which the mole ratio of ester to acylated acidic catalyst (calculated as S or P) is less than about 5:1, and in some embodiments less than about 2:1. The temperatures and pressures for vaporization of the ester can be in the range of about 80° C. to about 270° C. and pressure in the range of about 1 to about 500, in some embodiments about 5 to about 250 kilopascals absolute. Often at least two stages of vaporization are used, the last being under substantial vacuum, e. g, using a falling film evaporator or a wiped film evaporator.

The vaporization can be a flash vaporization or can be a fractional distillation. The vaporization can proceed in one or more steps as discussed above. The carboxylic anhydride can be selected to provide a higher or lower boiling carboxylic acid co-product than that of the product ester. The vaporization process can be designed and operated to separately recover that carboxylic acid co-product as is well known in the art. Where the carboxylic anhydride reacts to provide the same carboxylic acid as used in the esterification, it may be separated from the treated product and passed to the esterification of step (a),

The acylated acidic catalyst bottoms fraction can be treated to recover any residual ester. For instance, the bottoms fraction can be contacted with water to revert the ester to alcohol and carboxylic acid with the alcohol and/or carboxylic acid being separated, e.g., by vaporization for recycle to the esterification step.

### Catalyst Recovery

The residual liquid phase contains acylated catalyst and usually heavies and any ester or alcohol or carboxylic acid not removed by vaporization. Extraction by contact of the residual liquid phase with water provides an aqueous phase containing the acylated catalyst and a separate organic phase containing heavies and any substantially water insoluble alcohol or carboxylic acid. As discussed above, the contact with water can cause the ester to revert to alcohol and carboxylic acid. Where the acylated catalyst has acyl moieties such as triacetyl phosphate, hydrolysis generates the acidic catalyst and carboxylic acid. The acidic catalyst can be recycled, if desired, to the esterification. Where the carboxylic acid is the same as that used for the esterification, it is not essential to remove the carboxylic acid prior to recycling the catalyst. As water adversely affects the driving force to the ester, some or all of the water in the aqueous phase can be removed, e.g., by distillation, prior to introducing the catalyst into the esterification reactor.

Where the residual liquid phase contains sulfocarboxylic acid, hydrolysis does not occur, and the sulfocarboxylic acid, which itself is catalytic, remains as such in the aqueous phase. Frequently, where the acidic esterification catalyst comprises sulfocarboxylic acid, sulfuric acid is provided to the esterification in an amount sufficient to accommodate losses of sulfocarboxylic acid from the process, e.g., with the heavies portion of the residual liquid phase. The added sulfuric acid is converted to sulfocarboxylic acid by reaction with carboxylic anhydride in the processes of this disclosure.

### EXAMPLES

In the following examples all parts and percentages are by mass unless otherwise stated or obvious from the context.

### Example 1

To a liquid, 2000-gram, ethylene glycol diacetate sample containing about 1 mass percent 2-hydroxyethyl acetate and about 0.029 mass percent water is added 0.2 mass percent concentrated sulfuric acid (3.9 grams). This mixture is added to a 2-liter round bottom glass flask equipped with a heating mantle, and a magnetic stirrer bar having dimensions of 38 by 19 millimeters and operated at a rotation rate of about 500 revolutions per minute. The flask is attached to a glass condenser cooled by tap water (about 20° C.). Nitrogen is passed into the liquid at a rate of about 20 milliliters per minute (STP). The stirred mixture is heated, and at a temperature of about 120° C. about 46.2 grams of acetic anhydride are added to the mixture. The amount of acetic anhydride, on a stoichiometric basis, is in excess of that required to react with the water, 2-hydroxyethyl acetate and sulfuric acid groups. Approximately one minute after adding the acetic anhydride, a 6 milliliter sample is withdrawn while the mixture is about 125° C. and is analyzed to contain:
Ethylene glycol diacetate, mass percent: 95.6
Acetic anhydride, mass percent 0.0
2-Hydroxyethyl acetate, mass percent 0.037
Acetic acid, mass percent 2.55
Heavies, mass percent 0.0016
Heavies mass percent in these Examples was determined by gas chromatograph (“GC”), unless otherwise specified.

The mixture is further heated and reaches 170° C. after about 23 minutes after the acetic anhydride addition. The mixture is held at about 170° C. for about an additional 103 minutes. Samples of 6 milliliters each are periodically taken upon the mixture reaching 170° C. No statistically significant change in the composition is observed.

### Example 2

Example 1 is substantially repeated except 93 grams of acetic anhydride are added. The analysis after about a minute indicates the following composition:
Ethylene glycol diacetate, mass percent: 94.3
Acetic anhydride, mass percent 0.71
2-Hydroxyethyl acetate, mass percent 0.035
Acetic acid, mass percent 4.17
Heavies, mass percent 0.042

### Example 3

Example 1 is substantially repeated except a 200 gram sample of ethylene glycol diacetate is used and 0.02 mass percent of phosphoric acid is used instead of sulfuric acid. The analysis after about a minute indicates the following composition:
Ethylene glycol diacetate, mass percent: 97.1
Acetic anhydride, mass percent 0.13
2-Hydroxyethyl acetate, mass percent 0.0045
Acetic acid, mass percent 2.14
Heavies, mass percent 0.50

### Example 4

A sample of ethylene glycol diacetate treated with acetic anhydride in accordance with Example 1 is heated at 170° C. until about 95% of the ethylene glycol diacetate is evaporated. The residual liquid is contacted with an equal mass of water, and the mixture is vigorously mixed. Upon standing two distinct liquid layers are formed. The pH of the aqueous layer is determined to be about 1.36.

In comparison, a synthetic sample of ethylene glycol diacetate and sulfuric acid to match the mass ratio of acylated catalyst to ethylene glycol diacetate above is prepared. The sample is contacted with an equal mass of water and, and the mixture is vigorously mixed. Upon standing two distinct liquid layers are formed. The pH of the aqueous layer is determined to be about 0.45.

This comparison is repeated but using technical grade sulfoacetic acid (80% purity). The pH of the aqueous layer is determined to be about 0.74.

Although the disclosure has been described with reference to preferred embodiments, persons skilled in the art will recognize that changes may be made in form and detail without departing from the spirit and scope of the disclosed apparatus, systems and methods.

1. A process for the esterification of carboxylic acid having at least one carboxylic acid moiety with alcohol having at least one hydroxyl moiety to provide an ester, which process comprises:
a. contacting in a liquid phase the carboxylic acid and alcohol under esterification conditions including the presence of a catalytically-effective amount of homogeneous, acidic catalyst having an oxy-sulfur-containing or oxy-phosphorus-containing anion to whereby water and ester are produced;
b. removing water during step (a) to provide a reaction product containing less than about 5 mass percent water;
c. contacting the reaction product having water removed with carboxylic anhydride in an amount sufficient to convert at least about 98 percent of the acidic catalyst to acylated catalyst and provide a treated reaction product comprising ester, acylated catalyst and carboxylic acid co-product; and
d. separating ester from the treated reaction product by vaporization to provide a residual liquid phase comprising the acylated catalyst.

2. The process of claim 1 wherein in step (b), the water content is reduced to less than about 2 mass percent of the reaction product.

3. The process of claim 2 wherein the acidic catalyst comprises sulfuric acid and sulfocarboxylic acid.

4. The process of claim 2 wherein the acidic catalyst comprises one or more of sulfuric acid, sulfonic acid, sulfocarboxylic acid, phosphoric acid and phosphonic acid.

5. The process of claim 4 wherein the carboxylic acid has between about 2 and 6 carbon atoms.

6. The process of claim 5 wherein the carboxylic acid comprises acetic acid.

7. The process of claim 4 wherein the alcohol has up to 10 carbons.

8. The process of claim 7 wherein the alcohol has one or two hydroxyl moieties.

9. The process of claim 3 wherein carboxylic anhydride has carboxylic moieties each with between 2 and 18, carbon atoms.

10. The process of claim 9 wherein the carboxylic moieties of the carboxylic anhydride are the same as that of the carboxylic acid for the esterification.

11. The process of claim 4 wherein the carboxylic anhydride is provided in an amount of between 98 and 150 percent of that required on a stoichiometric basis to react with the hydroxyl groups, water and acid groups in the reaction product from step (b) having water removed.

12. The process of claim 4 wherein the separation of the ester in step (d) provides residual liquid phase in which the mole ratio of ester to acylated acidic catalyst (calculated as S or P) is less than about 10:1.

13. The process of claim 12 wherein the residual liquid phase of step (d) further contains heavies, and step (d) is conducted at a temperature in the range of about 80° C. to 270° C. and pressure in the range of about 1 to 500 kilopascals absolute.

14. The process of claim 13 wherein the residual liquid phase of step (d) is contacted with water to provide an aqueous layer containing acylated catalyst or hydrolysis product thereof and an organic liquid phase containing heavies.

15. The process of claim 14 wherein at least a portion of the acylated catalyst or hydrolysis product thereof is recycled to step (a).

16. A process for the esterification of acetic acid with ethylene glycol to provide ethylene glycol diacetate, which process comprises:
a. contacting in a liquid phase acetic acid and ethylene glycol under esterification conditions including the presence of a catalytically-effective amount of at least one of sulfuric acid and sulfoacetic acid whereby water, ethylene glycol diacetate and 2-hydroxyethyl acetate are produced;
b. removing water during step (a) to provide a reaction product containing less than about 2 mass percent water;
c. contacting the reaction product having water removed with acetic anhydride in an amount sufficient to convert at least about 98 percent of the total sulfuric acid and sulfoacetic acid to acylated catalyst and provide a treated reaction product comprising ethylene glycol diacetate, acylated catalyst and acetic acid; and
d. separating ethylene glycol diacetate from the treated reaction product by vaporization to provide a residual liquid phase comprising acylated catalyst.

17. The process of claim 16 wherein the residual liquid phase contains heavies, and the residual liquid phase is contacted with water to provide an aqueous liquid phase containing acylated catalyst and an organic phase containing heavies.

18. The process of claim 17 wherein at least a portion of the acylated catalyst in the aqueous phase is passed to step (a) as a co-catalyst.

19. A process for making sulfocarboxylic acid comprising contacting sulfuric acid and carboxylic anhydride under acylation conditions to produce sulfocarboxylic acid and carboxylic acid.

20. The process of claim 19 wherein the acylation conditions comprise a temperature between about 80° C. and 270° C.

21. The process of claim 19 wherein the carboxylic anhydride is acetic anhydride and the sulfocarboxylic acid is sulfoacetic acid.
